(19) United States
(12) Patent Application Publication
(10) Pub. No.: US 2024/0207214 A1
(43) Pub. Date: Jun. 27, 2024

(54) FATTY ACID LACTYLATES FOR USE IN TREATING RUMINANT ANIMALS

(71) Applicants: FORFARMERS CORPORATE SERVICES B.V., Lochem (NL); PURAC BIOCHEM B.V., Gorinchem (NL)

(72) Inventors: Adrianus Augustinus Maria Van Wesel, Made (NL); Hassan Zuhdi Taweel, Elst (NL); Evelien Johanna Marinus Jochems, Gorinchem (NL); Nicolaas Cornelis Maria Kors, Gorinchem (NL)

(21) Appl. No.: 18/556,309

(22) PCT Filed: Apr. 22, 2022
(86) PCT No.: PCT/EP2022/060700

§ 371 Date: Oct. 19, 2023

(30) Foreign Application Priority Data
Apr. 23, 2021 (NL) 2028053

(51) Int. Cl. A61K 31/23; A61K 31/231; A61P 1/14; A61P 15/14

(52) U.S. Cl. CPC A61K 31/23; A61K 31/231; A61P 1/14; A61P 15/14

(57) ABSTRACT

This disclosure relates to a method for reducing somatic cell count (SCC) in milk of a ruminant lactating animal, for increasing milk fat yield of a ruminant lactating animal, and/or for use in the prevention or treatment of mastitis in a ruminant animal, for increasing fecal consistency of a ruminant animal, for reducing methane production of a ruminant animal. In particular, this disclosure relates to a specified compound for use in the method, and to a nutrition composition for ruminant animals comprising the specified compound.

### CROSS REFERENCE TO RELATED APPLICATIONS

This application is a national phase entry under 35 U.S.C. § 371 of International Patent Application PCT/EP2022/060700, filed Apr. 22, 2022, designating the United States of America and published as International Patent Publication WO 2022/223787 A1 on Oct. 27, 2022, which claims the benefit under Article 8 of the Patent Cooperation Treaty to Dutch Patent Application Serial No. 2028053, filed Apr. 23, 2021.

### TECHNICAL FIELD

This disclosure relates to a method for reducing somatic cell count (SCC) in milk of a ruminant lactating animal, for increasing milk fat yield of a such ruminant lactating animal, and/or for use in the prevention of mastitis in a ruminant animal, for increasing fecal consistency of ruminant animals, for reducing methane production of such ruminant animals. In particular, this disclosure relates to a specified compound for use in the method, and to a nutrition composition for ruminant animals comprising the specified compound.

### BACKGROUND

An efficient digestion process and a good intestinal absorptive capacity of the animals are main factors that affect profitability on farm. Stimulating the intestinal function will increase absorption of nutrients and will lower nutrient excretion in the feces, leading to improved fecal consistency, as indicated by increased fecal scores, and higher nutrient utilization.

The digestive system is also responsible for the production of methane (CH₄), which is considered as a main greenhouse gas (GHG) that may contribute to the global warming phenomenon. In particular, ruminants have evolved a complex digestive tract and a symbiotic relationship with microbes that allow them to utilize poor quality feeds that are not suitable for human consumption. The downside of this otherwise useful symbiotic relationship is the production and emission of significant quantities of methane during the processes of feed fermentation. Methane in the rumen of ruminants is produced by methanogenic archaea that utilize CO₂ and H₂ as their primary substrates to generate CH₄.

Another aspect that affects profitability, in particular, of dairy farms, is udder health. Globally, 35% of culled/replaced cows in any herd are due to udder health issues. In addition, 90% of antibiotics used in the dairy industry are associated with udder health. Mastitis and high level of somatic cell count (SCC), lead to massive economic losses in the dairy industry. Improving udder health can improve animal welfare, reduce use of antibiotics, and improve milk yield, milk quality and thus economical returns of dairy farms.

CN102948652A discloses a cow food that may reduce mastitis, and which may increase total milk yield. Kim Eun Tae et al (Asian-Australas J Anim Sci. 2018 September; 31(9):1458-1463) discloses that supplementation of cow food with a surfactant may improve milk yield. WO2016126234 discloses the provision of compositions for improving milk yield in lactating ruminants such as cows. US2017333380 discloses the provision of animal feed compositions for milk cows. WO2013150058 discloses a method for improving economic performance in poultry husbandry. WO2009092787 discloses that lactylates may be useful for the prevention and treatment of infections caused by gram-positive bacteria in animals.

However, the prior art provides limited solutions for improving intestinal and/or udder health and there remains a need to develop new means for increasing fecal consistency, for reducing methane production, for improving udder health, and/or for increasing milk fat yield in ruminant animals.

### BRIEF SUMMARY

This disclosure provides a method and composition for animal feed for increasing fecal consistency of a ruminant animal, for reducing methane production of such a ruminant animal, for reducing somatic cell count (SCC) in milk of a ruminant lactating animal, for increasing milk fat yield of a ruminant lactating animal, and/or for use in the prevention of mastitis in such a ruminant animal. In accordance with this disclosure, use is made of a compound selected from:
lactylate in accordance with formula 1,

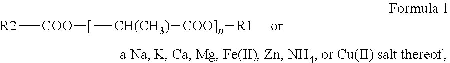

wherein in the above formulas R1 is selected from H, n stands for an integer with a value of 1-10, and R2 stands for a C1-C35 alkyl or alkenyl chain which may be branched or unbranched.

It was surprisingly found that administering a compound according to this disclosure (or derivatives thereof) stimulates intestinal function, increases rejuvenation and absorptive capacity of intestinal cells, and improves fecal consistency and fecal scores in ruminant animals. The addition of the compound to ruminants' diets can lead to a significant improvement in intestinal absorptive capacity, which will ultimately improve fecal consistency, fecal scores, and dry matter content. It was also found that administering a compound according to this disclosure (or derivatives thereof) can reduce methane emission from ruminant animals. For example, the addition of the compound to ruminants' diets can lead to a significant reduction in methane emission.

Equally surprisingly, it was found that administering a compound according to this disclosure (or derivatives thereof) can stimulate udder health, speed up udder recovery and reduce the incidence of mastitis in ruminant e.g., in a population of ruminant animals. For example, the addition of the compound to ruminants' diets can lead to a significant reduction in somatic cell count (SCC) and bacterial count (BC) in milk and to a reduction in the incidence and severity of mastitis in the herd. It was also found that administering a compound according to this disclosure (or derivatives thereof) can increase milk fat yield in ruminant lactating animals.

The applications according to this disclosure cover all domesticated ruminant and pseudo ruminant livestock such as cattle, goats, sheep, lamas and camels. A ruminant or pseudo-ruminant animal is any animal having three or more compartments to the stomach. Without being bound by any theory, the effects of the compound according to this disclosure may be caused by a different fermentation in the rumen, a change in protozoa population in the rumen and/or the very specific energy supply (e.g., more propionate, less acetate/butyrate) and specific fatty acids that the compound supplies to the intestinal cell wall and udder tissue.

### DETAILED DESCRIPTION

This disclosure provides for the use of a compound for increasing fecal consistency of a (healthy) ruminant animal and/or for reducing methane production of a ruminant animal, wherein the compound is selected from:
a lactylate in accordance with Formula 1,

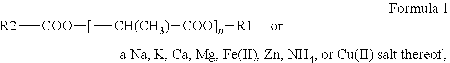

wherein in the above Formulae R1 is selected from H, n stands for an integer with a value of 1-10, and R2 stands for a C1-C35 alkyl or alkenyl chain which may be branched or unbranched. Preferably, the compound is a lactylate of formula 1 or a Na, K, Ca, Mg, Fe(II), Zn, NH₄, or Cu(II) salt thereof. In addition or alternatively, R2 is a C6-C18 alkyl or alkenyl chain. In addition or alternatively, n is 1, 2, or 3. The compound is preferably selected from one or more of lauroyl lactylate, myristoyl lactylate, cetyl lactylate or palmitoyl lactylate and the (sodium) salts thereof. In addition or alternatively, the compound may be selected from one or more of oleic acid lactylate or oleyl lactylate. The use of a lactylate of formula 1 or a salt thereof has been found to be preferred. Stearoyl lactylate is not particularly preferred and is preferably not used in this disclosure.

The use may be therapeutic (e.g., for the prevention or treatment of a medical condition or disease) or non-therapeutic (e.g., not for the prevention or treatment of a medical condition or disease). The use may involve administering the compound, e.g., an effective amount, to the ruminant animals, for example, as animal feed, or a supplement for animal feed. Accordingly, this disclosure provides for a method for increasing fecal consistency of a ruminant animal and/or for reducing methane production of a ruminant animal, the method comprising administering to the ruminant animal an effective amount of the compound (or any of its derivatives).

Preferably, the use according to this disclosure is not for preventing or treating an intestinal infection. In addition or alternatively, this disclosure may or may not be used for preventing or treating diarrhea. Likewise, the use according to this disclosure is preferably not for preventing or treating an infection by gram positive bacteria or an infection by gram negative bacteria.

Similarly, the compound according to this disclosure as defined above can be used to improve feed efficiency in a ruminant animal (e.g., as can be measured by g milk fat produced per kg (dry matter) feed intake for ruminant lactating animals), improve intestinal function, stimulate rejuvenation of intestinal cells (as can be determined e.g., by average cell age), for increasing (rumen or intestinal) production of propionate and/or for decreasing (rumen or intestinal) production of acetate/butyrate, and/or for changing protozoa population (in the rumen or intestine). The compound according to this disclosure as defined above can also be used for decreasing occurrence of disease in a population of more than 1, 10, 100, 500, 1000 of the ruminant animal, e.g., as determined over a period of at least 1, 2, 3, 4, 5, or 6 weeks, or at least 1, 2, 3, 4, 5, or 6 months, or at least 1, 2, 3, 4, 5, or 6 years. This may be affected by improving intestinal health by strengthening an intestinal wall in the ruminant animal. The disease may be any disease, but preferably not diarrhea and/or infections such as an infection by gram positive bacteria or an infection by gram negative bacteria.

Fecal consistency (or stool consistency) can be measured by any suitable means available in the art. For example, fecal consistency may be increased upon administering the compound according to this disclosure, in comparison to not administering the compound, e.g., as determined by a reduced water content in the stool. Alternatively, fecal consistency can be easily determined by scoring the fecal consistency according to the following scale:

| Score | Appearance | Description |
|---|---|---|
| 1 | Porridge, watery, green, | cattle are sick, stop eating, |
| | | eat a lot of fresh grass |
| 2 | Loose, not forming | just gave birth to a cow, eat |
| | | a lot of fresh grass |
| 3 | Pile shape, height 2.5~6.1 cm, double | High-yield cattle |
| | layer 2-4 concentric rings | |
| | Heap, 5~12 cm | Dry cows with low dietary |
| | | protein levels and high fiber |
| | | levels |
| 5 | Heap, 12 cm or more | Sick cow, and cows eating |
| | | roughage |

For example, the scale scores can be measured daily and averaged over a period of 14 days without administering the compound (for comparison), as well as over a period of 14 days while daily administering the compound. This can be done for one ruminant animal, or for a population of ruminant animals, for example, 2, 5, 10, 100, 500, 1000, or 5000 or more animals.

Also, methane production by an animal can be measured by any suitable means available in the art. For example, ruminal methane production may be decreased upon administering the compound according to this disclosure, in comparison to not administering the compound, e.g., as determined by a use of a respiration chamber, sulfur hexafluoride (SF6) tracer technique, breath sampling during milking or feeding, the GreenFeed system, or laser methane detector. Alternatively, methane production can be determined by an in vitro gas production technique (batch culture) (as described in e.g., Soliva et al., Letters in Applied Microbiology 2003, 37, 35-39). Briefly, e.g., rumen fluid, is obtained from the animal(s), e.g., 3 cows, and mixed with a buffer solution (CO₂-saturated bicarbonate/phosphate buffer, with buffer:rumen fluid 4:1, v:v) to a total of 25 ml to produce an inoculum for the in-vitro system, which is then transferred to sealed 125-mL flasks, in triplicate. The compound of this disclosure is then added (or a control:with inert compound, for comparison), e.g., 1 mg compound per ml inoculum, as well as a simulated animal ration. Then, the flasks are flushed with CO₂. After 24 h incubation period at 39° ° C. in a shaking incubator, the flasks can be removed from the incubator and put directly in an ice bath to stop the microbial activity. Total gas pressure can be measured, and the gas phase was sampled for gas composition analysis, e.g., by gas chromatography (GS) which allows quantification of H₂ and CH₄ in the incubation flasks and which can be performed using a micro-GC equipped with two gas chromatographic modules and a thermal conductivity detector (3000 micro-GC, Agilent, USA). Ethane (C₂H₆; 1 mL/flask) can be used as internal standard. For example, the in vitro gas production technique can be applied daily for daily obtained inoculum over a period of 14 days without the compound (for comparison), as well as with the compound. This can be done for inoculum obtained from one ruminant animal, or for a population of ruminant animals, for example, 2, 5, 10, 100, 500, 1000, or 5000 or more animals.

This disclosure also provides for the use of a compound for improving udder health of a ruminant lactating animal (e.g., as measured by reduced SCC and/or BC in milk of the animal, as also defined below), speed up udder recovery of a ruminant lactating animal, increase udder efficiency of a ruminant lactating animal, for reducing somatic cell count (SCC) in milk of a ruminant lactating animal, for reducing bacterial count (BC) in milk of a ruminant lactating animal and/or for increasing milk (fat) yield of a ruminant lactating animal, wherein the compound is selected from:
a lactylate in accordance with Formula 1,

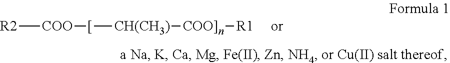

wherein in the above Formulae R1 is selected from H, n stands for an integer with a value of 1-10, and R2 stands for a C1-C35 alkyl or alkenyl chain which may be branched or unbranched. Preferably, the compound is a lactylate of formula 1 or a Na, K, Ca, Mg, Fe(II), Zn, NH₄, or Cu(II) salt thereof. In addition or alternatively, R2 is a C6-C18 alkyl or alkenyl chain. In addition or alternatively, n is 1, 2, or 3. The compound is preferably selected from one or more of lauroyl lactylate, myristoyl lactylate, cetyl or palmityl lactylate, oleyl lactylate and the (sodium) salts thereof. The use of a lactylate of formula 1 or a salt thereof has been found to be preferred. In addition or alternatively, the compound may be selected from one or more of oleic acid lactylate or oleyl lactylate. The use of a lactylate of formula 1 or a salt thereof has been found to be preferred. Stearoyl lactylate is not particularly preferred and is preferably not used in this disclosure.

The use may be therapeutic (e.g., for the prevention or treatment of a medical condition or disease) or non-therapeutic (e.g., not for the prevention or treatment of a medical condition or disease).

Somatic cell count (SCC) or bacterial count (BC) can be measured in any suitable way as available in the art. SCC may be measured using MIR (mid infrared spectroscopy), for example, as described in Rienesl et al, 2019, Acta Universitatis Agriculturae et Silviculturae Mendelianae Brunensis 67(5):1221-1226. Alternatively, a reliable direct method of measuring SCC or BC is by using an automatic cell counter; either by using a portable cell counter e.g., at the farm, or by sending milk samples to a laboratory for measurement in, for example, a Fossomatic cell counter. The advantage of an automatic cell counter is that it is objective and accurate. Generally, an individual cow SCC of 100,000 or less per ml milk indicates a healthy udder, where there are no significant production losses due to subclinical mastitis. A threshold SCC of 200,000 per ml or more may indicate that an animal has mastitis. BC may also be measured using standard plate count, or direct microscopic count.

Milk fat yield can be determined by any suitable means as available in the art. For example, milk fat yield can be determined by measuring the amount of fat in the (daily) milk yield. It is also possible to determine the fat corrected milk yield (milk yield adjusted to 3.5% fat).

For example, SCC, BC and/or milk fat yield can be measured daily and averaged for a period of 14 days without administering the compound to the animal (for comparison), as well as for a period of 14 days while daily administering the compound to the animal. This can be done for one animal, or for a population of animals, for example, 2, 5, 10, 100, 500 or more animals.

The use may involve administering the compound, e.g., an effective amount, to the ruminant animal, for example, as animal feed, or a supplement for animal feed. Accordingly, this disclosure provides for a method for improving udder health of a ruminant lactating animal, for reducing somatic cell count (SCC) in milk of a ruminant lactating animal, reducing bacterial count (BC) in milk of a ruminant lactating animal and/or for increasing milk fat yield of a ruminant lactating animal, the method comprising administering to the ruminant animal an effective amount of the compound (or any of its derivatives).

Preferably, the use according to this disclosure is not for preventing or treating an udder infection, e.g., caused by gram positive bacteria or gram negative bacteria.

Similarly, this disclosure provides for a compound for use in the prevention or treatment of mastitis in a ruminant (lactating) animal, wherein the compound is selected from:
a lactylate in accordance with Formula 1,

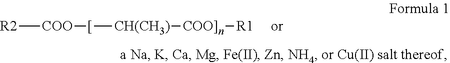

wherein in the above Formulae R1 is selected from H, n stands for an integer with a value of 1-10, and R2 stands for a C1-C35 alkyl or alkenyl chain which may be branched or unbranched. Preferably, the compound is a lactylate of formula 1 or a Na, K, Ca, Mg, Fe(II), Zn, NH₄, or Cu(II) salt thereof. In addition or alternatively, R2 is a C6-C18 alkyl or alkenyl chain. In addition or alternatively, n is 1, 2, or 3. The compound is preferably selected from one or more of lauroyl lactylate, myristoyl lactylate, cetyl or palmitoyl lactylate, oleyl lactylate and the (sodium) salts thereof. The use of a lactylate of formula 1 or a salt thereof has been found to be preferred. In addition or alternatively, the compound may be selected from one or more of oleic acid lactylate or oleyl lactylate. The use of a lactylate of formula 1 or a salt thereof has been found to be preferred. Stearoyl lactylate is not particularly preferred and is preferably not used in this disclosure.

The use may be therapeutic (e.g., for the prevention or treatment of a medical condition or disease) or non-therapeutic (e.g., not for the prevention or treatment of a medical condition or disease). Likewise, the use can be for decreasing occurrence of mastitis in a population of more than 1, 10, 100 of the ruminant animals. As mentioned herein, a threshold SCC of 200,000 per ml or more may indicate that a ruminant animal has mastitis.

The use may involve administering the compound, e.g., an effective amount, to the ruminant animal, for example, as animal feed, or a supplement for animal feed. Accordingly, this disclosure provides for a method the prevention of mastitis in a ruminant (lactating) animal, the method comprising administering to the ruminant animal an effective amount of the compound (or any of its derivatives).

Preferably, the use according to this disclosure is not for preventing or treating an udder infection. Likewise, the use according to this disclosure is preferably not for preventing or treating an infection by gram positive bacteria or an infection by gram negative bacteria.

The compound according to this disclosure (and for any use as defined above) may be comprised in an animal nutrition composition or animal feed composition for ruminants. An animal nutrition composition or animal feed composition may comprise wheat, starch, maize, sunflower meal, corn, cereals, barley, soybean meal, tapioca, citrus pulp, legumes, and/or beet pulp. In addition or alternatively, the compound may be in combination with one or more compounds selected from wheat, starch, maize, sunflower meal, corn, cereals, barley, soybean meal, tapioca, citrus pulp, legumes, and beet pulp. In addition or alternatively, the compound may be present on a support selected from vegetable fiber material, vegetable carbohydrates (such as cellulose) and mineral supports (such as silica, starch, gypsum, and lime). This provides a convenient way to obtain the compound in solid powdered form. The compound may also be in the form of a tablet or other shaped body known for provision of pharmaceutical components to animals.

In a preferred embodiment, the compound according to this disclosure (and for any use as defined above) is present in an animal nutrition composition for ruminants in an amount of 0.001 to 1 wt. %, more preferably 0.001 to 0.5 wt. %, based on the total weight of the animal nutrition composition. The compound may be added in a mixture with vegetable oil (e.g., a corn oil, soybean oil, or olive oil).

Preferably, the compound according to this disclosure (and for any use as defined herein) is applied in the absence of an inorganic acid selected from nitrogen, sulfur, and phosphorus-containing acids. In this way, the amount of non-dissociated lactic acid can be increased.

In all applications, preferably a mixture of lauroyl lactate and myristoyl lactylate is used. This may be in combination with a carrier as described earlier to obtain a lactylate powder. Lauroyl and myristoyl lactylate may also be applied in a liquid form which may be in further combination with oleyl lactylate as to improve both microbial efficacy as the physical handling of such blends and formulations.

In a preferred embodiment of this disclosure, and with respect to any use as disclosed herein, for example, for increasing fecal consistency of a ruminant animal, for reducing methane production of a ruminant animal, for increasing (rumen or intestinal) production of propionate and/or for decreasing (rumen or intestinal) production of acetate/butyrate, for changing protozoa population (in the rumen or intestine), for reducing somatic cell count (SCC) in milk of a ruminant lactating animal, for increasing milk fat yield of a ruminant lactating animal, and/or for use in the prevention or treatment of mastitis in a ruminant animal, R2 of the compound according to this disclosure may be an alkyl or alkenyl chain with 6-20 carbon atoms. More in particular, R2 may be an alkyl or alkenyl chain with 6-18 carbon atoms. In this embodiment, suitable substituents include groups with 6 carbon atoms (capronic), 8 carbon atoms (caprylic) 10 carbon atoms (capric acid), 12 carbon atoms (lauroyl), 14 carbon atoms (myristoyl), 16 carbon atoms (cetyl or palmityl), 18 carbon atoms (oleyl or stearyl). Mixtures of two or more compounds may also be used, for example, a mixture of C10/C12 lactylate, a mixture of C12/C14 lactylate, a mixture of C₁₀/C₁₂/C₁₄ lactylates, or a mixture of C12/C14/C18 lactylates. Where a salt is used, the use of a Na, K, Ca, or Mg salt may be particularly preferred.

The value for n is preferably in the range of 1-5. More in particular, n has a value of 1, 2, or 3.

The use of lauroyl lactylate, myristoyl lactylate, and their sodium salts is particularly preferred. In one embodiment, a mixture is used comprising 5-95 wt. % of lauroyl lactylate and 95-5 wt. % of myristoyl lactylate, or the sodium salt(s) of these compounds are used, more in particular, a mixture is used comprising 25-75 wt. %, more in particular, 40-60 wt. % of lauroyl lactylate, and 75-25 wt. %, more in particular, 40-60 wt. % of myristoyl lactylate, or the sodium salt(s) of these compounds.

The compound according to this disclosure, for any use as described above, may be administered to ruminant animals as a component of an animal feed composition. In the context of this disclosure, the term “animal nutrition” includes solid feed and liquid feed, such as drinking water. Thus, the composition may be administered to an animal as a solid or liquid component of a conventional animal feed composition or in their drinking water.

The compound may also be administered to the ruminant animal in a separate step, independent from the provision of a conventional animal feed composition.

The amount of the compound according to this disclosure, in particular, lactylate, administered to the ruminant animal may be such that it is effective for increasing fecal consistency of the ruminant animal, for reducing methane production of the ruminant animal, for increasing (rumen or intestinal) production of propionate and/or for decreasing (rumen or intestinal) production of acetate/butyrate, for changing protozoa population (in the rumen or intestine), for reducing somatic cell count (SCC) in milk of the ruminant lactating animal, for increasing milk fat yield of the ruminant lactating animal, and/or for use in the prevention or treatment of mastitis in the ruminant animal. Such an amount is suitably in the range from 0.0001-5% based on the total weight of the composition or feed fed to the ruminant animal. In a preferred embodiment, the amount may be in the range of 0.001 to 2%, based on the total weight of the composition or feed fed to the ruminant animal. Also, in one embodiment of this disclosure the amount may be in the range of 0.001 to 1 wt. %, more in particular, 0.001 to 0.5 wt. %, based on the total weight of the composition or feed to the animal. It is within the scope of the skilled person to determine the amount necessary.

For example, an ingredient composition comprising the compound according to this disclosure may comprise 10 to 40 wt. %, 25 to 35 wt. %. or 20 to 80 wt. % of the compound based on the total weight of the composition. Also foreseen is a stable stock solution comprising 0.4 to 12 wt. % of the ingredient composition, preferably from 0.4 to 10 wt. %, more preferably from 0.5 to 7.5 wt. %, most preferably from 0.5 to 5 wt. % based on the total weight of the stock solution. And also envisaged is a (liquid) animal feed comprising the ingredient composition wherein the final concentration of the ingredient composition in the feed is from 0.01 to 1.0 wt. %, preferably from 0.01 to 0.5, 0.6 or 0.7 wt. %, more preferably from 0.03 or 0.05 wt. % to 0.15, 0.20 or 0.25 wt. %, most preferably from 0.05 to 0.1 wt. % based on the total weight of the feed.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one.”

The following Examples illustrate the different embodiments of the this disclosure.

### EXPERIMENTAL SECTION

In this Experimental Section, various lactylates were tested, for example, C8 lactylate, C10 lactylate, C12 lactylate, C14 lactylate, C16 lactylate, C18:1 lactylate, mixture of 1:1 C10/C12 lactylate, mixture of 1:1 C12/C14 lactylate.

### Example 1—Lactylate (or Derivatives Thereof) as a Supplement to Lower Methane Emission in Animals

It was found that lactylates (or derivatives thereof) can reduce methane emission from ruminant animals. The application covers all domesticated ruminant and pseudo ruminant livestock having three or more compartments to their stomach such as cattle, goats, sheep, lamas and camels.

### Introduction

Methane (CH₄) has been considered as a main greenhouse gas (GHG) that may contribute to the global warming phenomenon. Ruminants have evolved a complex digestive tract and a symbiotic relationship with microbes that allow them to utilize poor quality feeds that are not suitable for human consumption. The downside of this otherwise useful symbiotic relationship is the production and emission of significant quantities of methane during the processes of feed fermentation. Methane in the rumen of ruminants is produced by methanogenic archaea that utilize CO₂ and H₂ as their primary substrates to generate CH₄. The aim of this study was to evaluate the efficacy of lactylates in reducing methane emission by ruminants.

### Materials and Methods

The well documented in vitro gas production technique (batch culture) (Soliva et al., Letters in Applied Microbiology 2003, 37, 35-39) was used in this study. The rumen fluid used in this incubation was obtained before the morning feeding from 3 rumen fistulated dairy cows on a basal diet composed of 50% maize silage and 50% wilted grass silage (on DM basis), supplemented with some straw and soybean meal. In addition, a balanced compound feed was fed to fulfill the animals' requirements for milk production. The rumen fluid of the three animals was mixed and used to prepare the inoculum. Incubations were performed in sealed 125-mL flasks, in triplicate. The supplements comprising the lactylates (in this case applied as ALOAPUR® powder as obtained from Purac/Corbion) at the level of 1 mg per ml buffered rumen fluid (see below), as well as the simulated dairy cow ration were added to the incubation flasks. On the day of the incubation, the flasks were sealed and flushed with CO₂. To each flask, 25 mL of CO₂-saturated bicarbonate/phosphate buffered rumen fluid was added (buffer:rumen fluid 4:1, v:v) before the onset of the in vitro incubations at 39° C. in a shaking incubator (Edmund Bühler Gmbh, Hechingen, Germany). After 24 h incubation period, the flasks were removed from the incubator and put directly in an ice bath to stop the microbial activity. Total gas pressure was measured, and the gas phase was sampled for gas composition analysis. Afterwards, pH was measured, and samples were collected for volatile fatty acids (VFA) analysis and protozoa numbers counting.

### Results

The efficacy of lactylates in reducing methane emission in ruminants in vitro is shown in Table 1. Adding lactylates to the fermentation media led to a significant (P<0.01) reduction in methane emission. Methane emission after 24 h of fermentation when lactylate was added to the flask was <10% of that of the control treatment, indicating that lactylates (1 mg/ml inoculum) managed to lower methane emission by >90%. Moreover, the addition of lactylate reduced protozoa count by 6% and shifted the fermentation toward more propionate and less acetate and butyrate. This indicates that lactylates will have a profound effect on ruminal fermentation that will lead to a significant reduction in methane emission.

TABLE 1
Effect of lactylates on in-vitro methane and VFA production
(μmol), VFA composition (mol/100 mol), and protozoa count

| | | Lactylates | | | |
|---|---|---|---|---|---|
| | Control | (1 mg/ml) | SEM¹ | % change | P value |
| Methane | 451 | 14 | 10.4 | −96% | 0.001 |
| (μmol/flask) | | | | | |
| Hydrogen | 0.66 | 11.4 | 1.68 | 1627% | 0.001 |
| (μmol/flask) | | | | | |
| Total VFA | 1190 | 930 | 35.4 | −21.8% | 0.001 |
| (μmol/flask) | | | | | |
| Acetate (M) | 61.9 | 44.3 | 1.25 | −28% | 0.001 |
| Propionate (M) | 22.1 | 52.1 | 1.31 | 240% | 0.001 |
| Butyrate (M) | 12.3 | 2.3 | 0.19 | −81% | 0.001 |
| pH | 6.64 | 6.76 | 0.032 | 1.8% | 0.02 |
| Protozoa | 5.08 | 4.79 | 0.075 | −5.7% | 0.04 |
| (Log10/mL) | | | | | |

¹Standard Error of Mean

### Conclusions

This study confirms the efficacy of Lactylate in reducing methane emission in ruminant animals. For example, the addition of lactylate to ruminants' diets can lead to a significant reduction in methane emission.

### Example 2—Lactylate (or its Derivatives) as a Supplement can Improve Udder Health, and Reduce Somatic Cell Counts (SCC) and Bacterial Counts (BC) in Milk of Ruminant Lactating Animals

It was found that lactylate (or its derivatives) can stimulate udder health, speed udder recovery and reduce the incidence of mastitis in ruminant animals. The application covers all domesticated ruminant and pseudo ruminant livestock such as cattle, goats, sheep, lamas and camels.

### Introduction

Udder health is of massive importance for the dairy industry. Globally, 35% of culled/replaced cows in any herd are due to udder health issues. In addition, 90% of the antibiotics used in the dairy industry are associated with udder health. Mastitis and high level of SCC lead to massive economic losses in the dairy industry. Improving udder health can improve dairy animal welfare, reduce use of antibiotics, and improve milk quality and economical returns of dairy farms. The aim of this study was to evaluate the efficacy of lactylates in improving udder health in ruminants.

### Materials and Methods

Two experiments were carried out to evaluate the efficacy of lactylates in stimulating udder health. The first experiment was conducted with goats and the second with dairy cows. In the first experiment, a goat farm was used. The farm had issues with udder health and had high levels of somatic cell count (SCC) and bacterial counts (BC) in milk delivered. Performance and udder health data were collected every 4 weeks from shortly before starting the experiment for a duration of 3 months. During the experiment all goats on the farm were offered a ration supplemented with lactylates (in this case applied as ALOAPUR® powder as obtained from Purac/Corbion) (added to the compound feed, 3.5 to 7 g per goat per day). During the 3-month study period, every 3 to 4 weeks, data on performance and udder health was collected. The 2^{nd} study was conducted on 7 dairy farms. The farms had an elevated level of SCC at the beginning of the study. Lactylates were supplemented to the ration of the cows on those farms via the compound feed (25 to 40 g per cow per day). The study lasted for 3 months. Performance and udder health data were collected at the beginning and every 4 weeks of the study.

### Results

The efficacy of Lactylates in reducing SCC and BC and the incidence of mastitis and in improving udder health in goats is shown in Table 2. Supplementing goats with Lactylates lowered SCC and BC in milk, indicating that Lactylates improved udder health.

TABLE 2
Effect of supplementing lactylates to dairy goats
on udder health, incidence of mastitis, somatic
Cell Count (SCC) and bacterial Count (BC) in milk

| | Before | After | After | After |
|---|---|---|---|---|
| | (wk −1) | (wk 4) | (Wk 8) | (Wk 12) |
| SCC (x1000/ml) | 203 | 157 | 145 | 127 |
| BC (x1000/ml) | 52 | 31 | 34 | 26 |

The efficacy of lactylates in reducing SCC and the incidence of mastitis and in improving udder health in dairy cows is shown in Table 3. Supplementing dairy cows with lactylates lowered SCC in milk, indicating that Lactylates improved udder health. Moreover, the number of dairy cows suffering from mastitis and incidence of new cases of mastitis was reduced when Lactylates was supplemented.

TABLE 3
Effect of supplementing lactylates to dairy cows on udder health,
incidence of mastitis, somatic Cell Count (SCC) in milk

| | Before | After | After |
|---|---|---|---|
| | (av 4 weeks) | (Wk 8) | (Wk 12) |
| SCC (x1000/ml) | 276 | 227 | 207 |
| % Cows >250 SCC | 22.1 | 20.7 | 17.6 |
| % New cows with high | 9.7 | 9.2 | 8.4 |
| SCC | | | |
| Mastitis cases (%) in 4- | 3.1 | 1.8 | 2.1 |
| week period | | | |

### Conclusions

The 2 studies confirm the efficacy of lactylate in improving udder health in both dairy goats and dairy cows. For example, the addition of Lactylate to ruminants' diets can lead to a significant reduction in SCC and BC in milk and to a reduction in the incidence and severity of Mastitis in the herd.

### Example 3—Lactylate (or Derivatives Thereof) as a Supplement to Stimulate Rejuvenation of Intestinal Cells, Improve Fecal Consistency and Dry Matter and Increase Milk Fat Yield in Ruminant Lactating Animals

It was found that the use of lactylate (or derivatives thereof) stimulates intestinal function, rejuvenation and absorptive capacity, and improves fecal consistency and fecal scores in ruminant animals. The application covers all domesticated ruminant and pseudo ruminant livestock such as cattle, goats, sheep, lamas and camels.

### Introduction

Feed efficiency is the main factor that affects profitability on farm. Improving the digestion process, and the digestive and absorptive capacity of the intestine plays a major role in improving feed efficiency. Stimulating the rejuvenation and function of intestinal cells will lower nutrients excretion in the feces and increase absorption of nutrients, leading to improved fecal scores, dry matter content and consistency and higher nutrient utilization. Additionally, stimulating and increasing milk components, especially milk fat % and yield will lead, if feed intake remains the same, to improvements in feed efficiency. The aim of this study was to evaluate the efficacy of lactylate in improving fecal consistency, dry matter content and scores, and increasing milk fat yield in ruminants.

### Materials and Methods

Two experiments were carried out to evaluate the efficacy of lactylate in stimulating intestinal absorptive capacity and improving fecal consistency, dry matter content and scores and milk fat yield. The first experiment was conducted with goats and the second with dairy cows. The focus of the first experiment was the absorptive capacity and fecal consistency and scores, while the 2^{nd} experiment focused on the effect on milk fat yield. In the first experiment, a goat farm was used. The farm had issues with fecal consistency, having at least 30 goats per pen with low fecal consistency. Fecal consistency and scores per pen were recorded 2 times per week from the start of the experiment for a period of 3 months. The goats in the farm were supplemented with lactylates (in this case applied as ALOAPUR® powder as obtained from Purac/Corbion) (added to the compound feed) at the rate of 3.5 to 7 g per goat per day. The 2nd study focused on the effect on milk fat yield and was conducted at a dairy research farm. The herd (120 cows) was divided into 2 groups blocked on days in milk, milk yield and milk fat %. One group was supplemented with 50 g of lactylates per cow per day while the other group served as a control. The ration for both groups was similar and was balanced on energy, protein and minerals and vitamins. The experiment lasted for 9 week. Performance data was collected/recorded on weekly basis.

### Results

The efficacy of lactylate in improving fecal consistency of ruminants is shown in Table 4. Supplementing goats with lactylates reduced the number of goats with low fecal consistency per pen significantly. Fecal scores improved as the experiment progressed indicating that the longer the goats were receiving the supplement the better (higher) their fecal consistency became.

TABLE 4
Effect of supplementing lactylates to dairy goats
on fecal scores, dry matter and consistency

| | Before | After | After | After | |
|---|---|---|---|---|---|
| | (wk −1) | (wk 4) | (Wk 8) | (Wk 12) | P value |
| Number of goats | 29 | 15 | 8 | 4 | 0.01 |
| with low fecal | | | | | |
| consistency scores | | | | | |
| <2/pen | | | | | |

The effect of supplementing dairy cows with lactylates on milk fat yield is shown in Table 5. Adding 50 g/c/d to the ration increased milk fat % (by 0.11%, 3% relative increase) and fat yield (by 63 g/c/d, 4% relative increase).

TABLE 5
Effect of supplementing lactylates to dairy
cows on milk yield and composition

| | Control | Lactylate | P value |
|---|---|---|---|
| Milk (kg/d) | 38.2 | 38.5 | 0.56 |
| Fat (%) | 4.43 | 4.54 | 0.12 |
| Protein (%) | 3.59 | 3.60 | 0.43 |
| Fat (g/d) | 1647 | 1710 | 0.10 |
| Protein (g/d) | 1350 | 1363 | 0.32 |
| Urea (mg/dl) | 24.7 | 25.7 | 0.65 |

### Conclusions

The 2 experiments with dairy goats and dairy cows confirm the efficacy of lactylate in improving fecal consistency, scores, and dry matter, and in increasing milk fat % and fat yield. The addition of lactylate to ruminants' diets can lead to a significant improvement in intestinal absorptive capacity, which will ultimately improve fecal consistency, scores, and dry matter and increase milk fat % and fat yield in ruminants.

### Example 4—Comparison of Lactylates

This Examples shows a comparison of using different lactylates in a diet for 70 ruminants (7 groups of 10 ruminants, including control group), and the effect on methane emission, SCC, mastitis, fecal consistency, and milk fat yield. Methane emission is determined as described in Example 1; SCC, BC and mastitis effects are determined as described in Example 2. Effect on fecal consistency and effect on milk yield is determined as described in Example 3. The different lactylates are stearoyl lactylate, lauroyl lactylate, myristoyl lactylate, cetyl lactylate, palmitoyl lactylate and oleic lactylate. As control, the same diet without lactylates is supplied.

TABLE 6
Comparison of lactylates relative to control group

| | Stearoyl | Lauroyl | Myristoyl | Cetyl | Palmitoyl | Oleic |
|---|---|---|---|---|---|---|
| | lactylate (10 | lactylate (10 | lactylate (10 | lactylate (10 | lactylate (10 | lactylate (10 |
| | ruminants) | ruminants) | ruminants) | ruminants) | ruminants) | ruminants) |
| Effect on | Methane | Methane | Methane | Methane | Methane | Methane |
| methane | emission not | emission | emission | emission | emission | emission |
| emission | significantly | reduction | reduction | reduction | reduction | reduction |
| | different | by >90% | by >90% | by >90% | by >90% | by >90% |
| | from control | relative to | relative to | relative to | relative to | relative to |
| | | control | control | control | control | control |
| Effect on | SCC, BC and | SCC, BC in | SCC, BC in | SCC, BC in | SCC, BC in | SCC, BC in |
| SCC, BC | mastitis | milk reduced | milk reduced | milk reduced | milk reduced | milk reduced |
| and mastitis | incidence not | by >20%, | by >20%, | by >20%, | by >20%, | by >20%, |
| | significantly | incidence of | incidence of | incidence of | incidence of | incidence of |
| | different | mastitis | mastitis | mastitis | mastitis | mastitis |
| | from control | reduced | reduced | reduced | reduced | reduced |
| | | by >25% | by >25% | by >25% | by >25% | by >25% |
| | | relative to | relative to | relative to | relative to | relative to |
| | | control | control | control | control | control |
| Effect on | Number of | Number of | Number of | Number of | Number of | Number of |
| fecal | ruminants | ruminants | ruminants | ruminants | ruminants | ruminants |
| consistency | with low | with low | with low | with low | with low | with low |
| score | fecal | fecal | fecal | fecal | fecal | fecal |
| | consistency | consistency | consistency | consistency | consistency | consistency |
| | not | reduced | reduced | reduced | reduced | reduced |
| | significantly | by >50% | by >50% | by >50% | by >50% | by >50% |
| | different | relative to | relative to | relative to | relative to | relative to |
| | from control | control | control | control | control | control |
| Effect on | Average milk | Average milk | Average milk | Average milk | Average milk | Average milk |
| milk fat | fat yield not | fat yield | fat yield | fat yield | fat yield | fat yield |
| yield | significantly | increased | increased | increased | increased | increased |
| | different | by >10% | by >10% | by >10% | by >10% | by >10% |
| | from control | relative to | relative to | relative to | relative to | relative to |
| | | control | control | control | control | control |

Table 6 shows that the effect of diet supplementation of lauroyl lactylate, myristoyl lactylate, cetyl lactylate, palmitoyl lactylate or oleic lactylate is very similar and leads to very good results, whereas the effect of stearoyl lactylate is limited. It is expected that the putative results of Table 6 can be obtained in large ruminant populations.

1. A method of increasing milk fat yield of a ruminant lactating animal, the method comprising:
administering a lactylate to the animal,
wherein the lactylate is selected from one or more of lauroyl lactylate, myristoyl lactylate, cetyl lactylate, palmitoyl lactylate or oleic lactylate and the sodium salts thereof.

2. A method of preventing or treating mastitis in a ruminant animal and/or for reducing somatic cell count (SCC) in milk of a ruminant lactating animal, the method comprising:
administering a lactylate to the animal,
wherein the lactylate is selected from one or more of lauroyl lactylate, myristoyl lactylate, cetyl lactylate, palmitoyl lactylate or oleic lactylate and the sodium salts thereof.

3. A method of increasing fecal consistency of a healthy ruminant animal and/or for reducing methane production of a ruminant animal, wherein the lactylate is selected from
a lactylate of Formula 1, 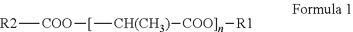
or a Na, K, Ca, Mg, Fe(II), Zn, NH₄, or Cu(II) salt thereof,
wherein in Formulae R1 is selected from H, n stands for an integer with a value of 1-10, and R2 stands for a C1-C35 alkyl or alkenyl chain which may be branched or unbranched,
wherein if the method is for increasing fecal consistency of a healthy ruminant animal, the lactylate is selected from one or more of lauroyl lactylate, myristoyl lactylate, cetyl lactylate, palmitoyl lactylate or oleic lactylate and the sodium salts thereof.

4. The method according to claim 3, wherein the method further increases rejuvenation of intestinal cells in the ruminant animal.

5. The method according to claim 3, wherein the method further decreases occurrence of disease in a population of more than 10 of said ruminant animals.

6. The method according to claim 1, wherein the method further reduces bacterial count (BC) in milk of said ruminant lactating animal.

7. The method according to claim 1, wherein the use is not for preventing or treating an infection.

8. The method according to claim 1, wherein the compound is comprised in a ruminant nutrition composition administered to the animal.

9. The method according to claim 1, wherein the ruminant lactating animal is selected from cattle or goat.

10. The method according to claim 4, wherein the method further decreases occurrence of disease in a population of more than 10 of the ruminant animals.

11. The method according to claim 1, wherein the method further reduces bacterial count in milk of the ruminant lactating animal.

12. The method according to claim 2, wherein the method is not for preventing or treating an infection.

13. The method according to claim 3, wherein the method is not for preventing or treating an infection.

14. The method according to claim 2, wherein the compound is comprised in a ruminant nutrition composition administered to the ruminant animal.

15. The method according to claim 3, wherein the compound is comprised in a ruminant nutrition composition administered to the ruminant animal.

16. The method according to claim 1, wherein the ruminant lactating animal is selected from cattle or goat.

17. The method according to claim 2, wherein the ruminant animal is selected from cattle or goat.

18. The method according to claim 3, wherein the ruminant animal is selected from cattle or goat.
